(19) United States
(12) Patent Application Publication
(10) Pub. No.: US 2026/0355456 A1
(43) Pub. Date: Nov. 12, 2026

(54) Urinary Catheter Devices and Methods

(71) Applicant: C. R. Bard, Inc., Franklin Lakes, NJ (US)

(72) Inventors: Pooja Bhuvanesh Kulkarni, Pune (IN); Sudev Geetha Surendran, Kozhikode District (IN); James David Hughett, Sr., Monroe, GA (US); Ronald N. Legaspi, Alpharetta, GA (US)

(21) Appl. No.: 19/131,909

(22) PCT Filed: Nov. 21, 2022
(86) PCT No.: PCT/US2022/050645

§ 371 Date: May 21, 2025

(51) Int. Cl. A61M 25/00; A61M 25/06; A61M 39/20

(52) U.S. Cl. CPC A61M 25/0017; A61M 25/0097; A61M 39/20; A61M 2025/0047; A61M 2025/0681; A61M 2205/0216

(57) ABSTRACT

A urinary catheter (100) generally configured for self-deployment. The catheter includes a lubricant (108) disposed within the catheter lumen. The catheter further includes a sheath (125) covering the catheter tube, where a lumen of the sheath also contains the lubricant. The catheter includes an engagement member at the proximal end that is selectively transitionable between a closed state and an open state. Various embodiments of caps (160) that define the engagement member are disclosed. The cap may also be retained in the open position so as to be positioned away of a urine exiting the catheter lumen during use.

### BACKGROUND

Urinary incontinence causes a person to lose control over their bladder resulting in loss of large or small quantities of urine. This can be caused due to multiple reasons. A paraplegic or tetraplegic user loses the control of their bladder and they need to empty their bladder using urinary catheters. These can be indwelling (the catheter is placed for a longer duration inside the bladder and attached to a urine bag to collect the urine) or they can be intermittent (the catheter is periodically inserted into the bladder and the urine is drained from it either directly in appropriate drainage area or inside a urine bag). Intermittent catheters are preferred in the cases where the user can self-catheterize or wishes to not be seen with a urine bag. The intermittent catheters allow the user a freedom of mobility.

A catheter can be pre-lubricated. In some cases, the lubricant can spill while opening the package which can soil or stain the user's clothing. Similarly, residual urine within the catheter lumen can exit the catheter lumen and soil or stain the user's clothing. In some cases, urinary catheters include a packaging shape that is not conducive to carry on one's person, such as within a pocket of clothing.

Disclosed herein are urinary catheters and methods of catheterization that address the items described above.

### SUMMARY

Briefly summarized, disclosed herein is a urinary catheter that, according to some embodiments, includes: (i) a catheter tube defining a catheter lumen extending between distal end and a proximal end, where the catheter tube is configured for insertion into a patient bladder; (ii) a sheath defining a sheath lumen extending along the catheter tube, where the catheter tube disposed within the sheath lumen; (iii) an outlet hub coupled with the catheter tube at the proximal end. The outlet hub includes a hub body defining a hub chamber, and the hub chamber defines the catheter lumen extending through the outlet hub. The catheter further includes an engagement member coupled with the outlet hub, where the engagement member is selectively transitionable between (i) a closed state wherein a fluid is prevented from exiting the catheter lumen at the proximal end and (ii) an open state wherein the fluid is allowed to exit the catheter lumen at the proximal end.

In some embodiments, the catheter further includes a lubricant disposed within the catheter lumen. In some embodiments, the lubricant is further disposed within the sheath lumen along an exterior of the catheter tube. In some embodiments, the fluid includes the lubricant.

In some embodiments, the engagement member includes a lid coupled with the outlet hub via a hinge. The lid is rotatable between a closed position defining the closed state and an open position defining the open state, where the lid extends across a proximal end of the outlet hub in the closed position, and where the lid is rotated away from the proximal end of the outlet hub in the open position.

In some embodiments, when the lid is in the open position, the lid is rotated at least 90 degrees away from the closed position. In some embodiments, when the lid is in the open position, the lid is rotated at least 180 degrees away from the closed position. In some embodiments, the lid is rotatably biased toward the open position via the hinge.

In some embodiments, the lid includes a first protrusion extending away from a circumferential edge of the lid, and the outlet hub includes a corresponding second protrusion extending away from a side wall of the outlet hub, where the corresponding second protrusion is positioned adjacent the hinge. An interference between the corresponding second protrusion and the first protrusion defines a detent to maintain the lid in the open position.

In some embodiments, the outlet hub includes a protrusion extending away from a side wall of the outlet hub, and the lid includes a protrusion receiving member disposed on a top side of the lid, where the receiving member is configured to inhibit separation of the protrusion from the protrusion receiving member in the absence of a separating force. The protrusion is inserted into the protrusion receiving member to maintain the lid in the open position.

In some embodiments, the lid includes a plug disposed on an underside of the lid, and at least a portion of the plug is disposed within the hub chamber when the lid is in the closed position. In some embodiments, the plug is formed of an elastomeric material.

In some embodiments, the engagement member includes a lid coupled with the outlet hub via a tether. The lid is positionable between a closed position defining the closed state and an open position defining the open state, where the lid extends across a proximal end of the outlet hub in the closed position, and where the lid is positioned away from the proximal end of the outlet hub in the open position. The tether biases the lid away from the closed position toward the open position.

In some embodiments, the engagement member includes a plug cap disposed within the hub chamber, where the plug cap includes a fluid flow path extending through the plug cap between an underside of the plug cap and one or more side ports located along a cylindrical side wall of the plug cap. The plug cap is positionable between a closed position defining the closed state and an open position defining the open state, where the one or more side ports are disposed within the hub chamber when plug cap is in the closed position such that a side wall of the outlet hub sealably covers the one or more side ports, and where the one or more side ports are disposed proximally beyond the side wall of the outlet hub when the plug cap is in the open position.

In some embodiments, the plug cap is coupled with the outlet hub so as to be tiltable with respect to the outlet hub about an axis of rotation extending laterally through the plug cap and the outlet hub. The plug cap is oriented such that a proximal end face of the plug cap is perpendicular to a longitudinal axis of the outlet hub when the plug cap is in the closed position, and the plug cap is tilted such that the proximal end face is rotated away from the perpendicular orientation when the plug cap is in the open position In some embodiments, the plug cap is longitudinally positionable between the closed position and the open position.

In some embodiments, the outlet hub includes a ring extending circumferentially around an exterior of the hub body, and the ring is operatively coupled between the plug cap and the hub body such that (i) rotation of the ring in one direction with respect to the hub body causes the plug cap to proximally displace from the closed position to the open position, and (ii) rotation of the ring in the opposite direction with respect to the hub body causes the plug cap to distally displace from the open position to the closed position.

In some embodiments, the outlet hub includes a latching mechanism operatively coupled between the plug cap and the hub body, such that the latching mechanism positions the plug cap at the open position when a distally oriented force is (i) manually applied to the plug cap and (ii) released from the plug cap when the plug cap is in the closed position, and the latching mechanism positions the plug cap at the closed position when the distally oriented force is (i) manually applied to the plug cap and (ii) released from the plug cap when the plug cap is in the open position.

Also disclosed herein is a method for draining urine from a patient bladder. According to some embodiments, the method includes providing a catheter having a catheter tube configured for insertion into the patient bladder, where the catheter tube defines a catheter lumen. The method further includes transitioning an engagement member of the catheter from a closed state to an open state, where the engagement member is configured to prevent fluid from exiting a proximal end of the catheter lumen in the closed state through an outlet hub coupled with the catheter tube at the proximal end. The method further includes: (i) draining a lubricant from the catheter lumen; (ii) inserting the catheter tube into the patient bladder to establish a flow of urine through the catheter lumen, and (iii) transitioning the engagement member from the open state to the closed state.

In some embodiments of the method, the catheter further includes a sheath extending along the catheter tube, where the catheter tube is disposed within a lumen of the sheath, where a portion of the lubricant is disposed within the lumen of the sheath, and where the portion of the lubricant contacts an outer surface of the catheter tube.

In some embodiments of the method, the engagement member includes a lid coupled the outlet hub via a hinge. The lid is rotatable between a closed position defining the closed state and an open position defining the open state, where the lid extends across a proximal end of the outlet hub in the closed position, and where the lid is rotated away from the proximal end of the outlet hub in the open position. Transitioning the engagement member from the closed state to the open state includes rotating the lid at least 90 degrees away from the closed position.

In some embodiments, the lid includes a first protrusion extending away from a circumferential edge of the lid, and the outlet hub includes a corresponding second protrusion extending away from a side wall of the outlet hub, where the corresponding second protrusion positioned adjacent the hinge, and where an interference between the corresponding second protrusion and the first protrusion defines a rotational detent for the lid. In such embodiments of the method, transitioning the engagement member from the closed state to the open state includes rotating the lid beyond the detent to secure the lid in the open state.

In some embodiments of the method, the outlet hub includes a protrusion extending away from a side wall of the outlet hub, and the lid includes a protrusion receiving member disposed on a top side of the lid, where the protrusion receiving member is configured to inhibit separation of the protrusion from the protrusion receiving member in the absence of a separating force. In such embodiments of the method, transitioning the engagement member from the closed state to the open state includes rotating the lid to cause the protrusion to enter the receiving member to constrain the lid in the open position.

In some embodiments of the method, the engagement member includes a lid coupled with the outlet hub via a tether. The lid is positionable between a closed position defining the closed state and an open position defining the open state, where the lid extends across a proximal end of the outlet hub in the closed position, and where the lid is positioned away from the proximal end of the outlet hub in the open position. The tether biases the lid away from the closed position toward the open position, and in such embodiments of the method, transitioning the engagement member from the closed state to the open state includes (i) displacing the lid away from the closed position and (ii) allowing the tether to displace the lid toward the open position.

In some embodiments of the method, the engagement member includes a plug cap disposed within a chamber of the outlet hub, where the plug cap includes a fluid flow path extending through the plug cap between an underside of the plug cap and one or more side ports located along a cylindrical side wall of the plug cap. The plug cap is positionable between a closed position defining the closed state and an open position defining the open state, where the one or more side ports are disposed within the hub chamber when plug cap is in the closed position such that a side wall of the outlet hub sealably covers the one or more side ports, and where the one or more side ports are disposed proximally beyond the side wall of the outlet hub when the plug cap is in the open position. In such embodiments of the method, transitioning the engagement member from the closed state to the open state includes positioning the plug cap away from the closed position to the open position.

In some embodiments of the method, the plug cap is coupled with outlet hub so as to be tiltable with respect to the outlet hub about an axis of rotation extending laterally through the plug cap and the outlet hub, and the plug cap is oriented such that a proximal end face of the plug cap is perpendicular to a longitudinal axis of the outlet hub when the plug cap is in the closed position. In such embodiments of the method, transitioning the engagement member from the closed state to the open state includes tilting the plug cap away from the perpendicular orientation.

In some embodiments of the method, the plug cap is longitudinally positionable between the closed position and the open position.

In some embodiments of the method, the outlet hub includes a ring extending circumferentially around an exterior of the hub body, where ring is operatively coupled between the plug cap and the hub body such that rotation of the ring with respect to the hub body causes the plug cap to proximally displace from the closed position to the open position. In such embodiments of the method, transitioning the engagement member from the closed state to the open state includes rotating the ring.

In some embodiments of the method, the outlet hub includes a latching mechanism operatively coupled between the plug cap and the hub body, where latching mechanism is configured to position the plug cap at the open position when a distally oriented force is (i) manually applied to the plug cap and (ii) released from the plug cap when the plug cap is in the closed position. In such embodiments of the method, transitioning the engagement member from the closed state to the open state includes (i) applying the distally oriented force to the plug cap and (ii) allowing the latching mechanism to proximally displace the plug from the closed position to the open position.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustration of a urinary catheter in a packaging state, in accordance with some embodiments;

FIG. 1B is an illustration of the urinary catheter of FIG. 1A in an unpackaged state, in accordance with some embodiments;

FIG. 1C is a detailed illustration of a proximal end portion of the urinary catheter of FIG. 1A where the cap is in a closed position, in accordance with some embodiments;

FIG. 1D is a detailed illustration of a proximal end portion of the urinary catheter of FIG. 1A where the cap is in an open position, in accordance with some embodiments;

FIG. 1E is a detailed illustration of the proximal end portion of the urinary catheter of FIGS. 1C-1D where the cap is in a closed position, in accordance with some embodiments;

FIG. 2A is an illustration of a second embodiment of the outlet hub of the urinary catheter of FIG. 1A, where the cap is in a closed position, in accordance with some embodiments;

FIG. 2B is an illustration of the outlet hub of FIG. 2A where the cap is in an open position, in accordance with some embodiments;

FIG. 3A is an illustration of a third embodiment of the outlet hub of the urinary catheter of FIG. 1A, where the cap is in a closed position, in accordance with some embodiments;

FIG. 3B is an illustration of the outlet hub of FIG. 3A where the cap is in an open position, in accordance with some embodiments;

FIG. 4A is an illustration of a fourth embodiment of the outlet hub of the urinary catheter of FIG. 1A, where the cap is in a closed position, in accordance with some embodiments;

FIG. 4B is an illustration of the outlet hub of FIG. 4A where the cap is in an open position, in accordance with some embodiments;

FIG. 5A is an illustration of a fifth embodiment of the outlet hub of the urinary catheter of FIG. 1A, where the cap is in a closed position, in accordance with some embodiments;

FIG. 5B is an illustration of the outlet hub of FIG. 5A where the cap is in an open position, in accordance with some embodiments;

FIG. 6A is an illustration of a sixth embodiment of the outlet hub of the urinary catheter of FIG. 1A, where the cap is in a closed position, in accordance with some embodiments;

FIG. 6B is an illustration of the outlet hub of FIG. 6A where the cap is in an open position, in accordance with some embodiments;

FIG. 7A is an illustration of a seventh embodiment of the outlet hub of the urinary catheter of FIG. 1A, where the cap is in a closed position, in accordance with some embodiments;

FIG. 7B is an illustration of the outlet hub of FIG. 7A where the cap is in an open position, in accordance with some embodiments;

FIG. 8A is an illustration of an eighth embodiment of the outlet hub of the urinary catheter of FIG. 1A, where the cap is in a closed position, in accordance with some embodiments;

FIG. 8B is an illustration of the outlet hub of FIG. 8A where the cap is in an open position, in accordance with some embodiments; and

FIG. 9 is an illustration of a ninth embodiment of the outlet hub of the urinary catheter of FIG. 1A, where the hub includes a seal in the form of a film, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

The phrases “connected to,” “coupled with,” and “in communication with” refer to any form of interaction between two or more entities, including but not limited to mechanical, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

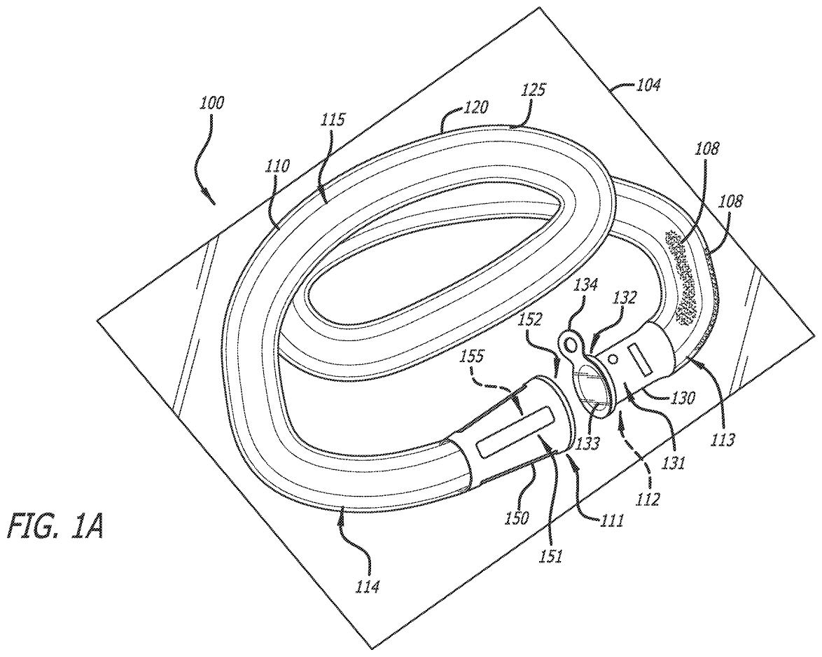

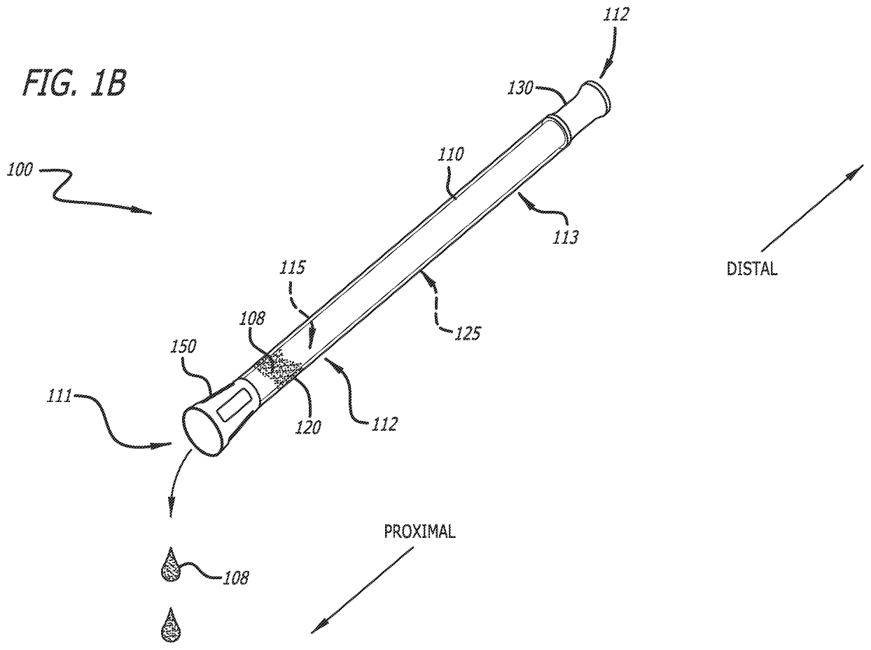

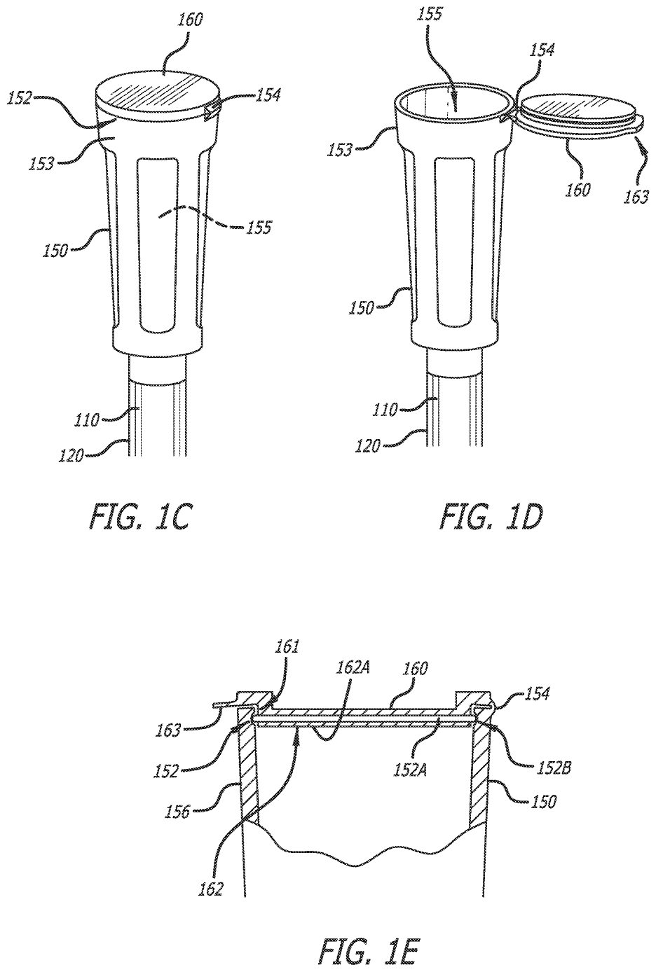

FIGS. 1A-1D illustrate a first embodiment of a urinary catheter in various states of use. The urinary catheter (catheter) 100 is generally configured for self-deployment by a user (also referred to herein as a patient) to drain the user's bladder of urine. The catheter 100 as illustrated is generally configured for use by a male patient. However, the catheter 100 may be configured for use by a female patient without departing from the features and functional aspects of the catheter 100 as shown and described below. The catheter 100 may be used by the patient in various settings, such as at a medical facility, at a home, or at a public restroom, for example.

The catheter 100 is generally configured to be used by the patient multiple times during a day. More specifically, the patient may deploy multiple catheters 100 (one at a time) during the day. As such, the patient my keep and carry one or more catheters 100 on his person, such as in a pocket of clothing or a purse, for example. As such, the catheter 100 may be disposed in packaging state having a compact shape consistent with placement of the catheter 100 within the pocket of clothing or purse. The catheter 100 is also configured to maintain a sterility of the catheter 100 in the packaging state.

In some instances, the user may be concerned about soiling clothing during the urine draining process, via the urine or a lubricant of the catheter, for example. Hence, the catheter 100 is configured for deployment while also inhibiting the spilling of a lubricant of the catheter 100 on clothing or other items during use. Furthermore, the catheter 100 is generally configured to contain any residual urine within the catheter tubing after use.

As the catheter 100 is configured for self-deployment, the catheter 100 may be configured for ease in handling (i.e., gripping, orienting, guiding, or applying a force, etc.) by a user during deployment. As such, the catheter 100 may be configured for deployment by users having limited dexterity.

FIG. 1A illustrates a perspective front view of the catheter 100 in a packaging state. The catheter 100 may be disposed in the packaging state before use. The catheter 100 may be disposed within an outer package 104, such as a bag or pouch, for example. In some embodiments, the outer package 104 may be resealable. In other words, the outer package 104 may be configured to (i) sealably contain the catheter 100 before use and (ii) sealably contain the catheter 100 after use.

The catheter 100 generally includes a catheter tube 110 having a proximal end 111 and a distal end 112. The catheter tube 110 may be formed of any suitable thermoplastic material via an extrusion process. A catheter lumen 115 extends along the catheter tube 110, where the catheter lumen 115 is sized to facilitate urine flow therethrough from the distal end 112 to the proximal end 111 during use. The catheter tube 110 includes an inlet section 113 extending proximally away from the distal end 112 is configured for insertion into and advancement along a urethra of the user to the patient's bladder. As such, the inlet section 113 defines a length sufficient to extend from the end of a penis to the bladder of the patient. Similarly, the inlet section defines an outside diameter compatible with placement within the urethra. The catheter tube 110 also includes an outlet section 114 extending distally away from the proximal end 111. The outlet section 114 is configured for use outside of the patient body and defines a length compatible with placement of the proximal end 111 over a urine receptacle, such as a toilet, for example, during use.

The catheter tube 110 includes an inlet collar 130 coupled with the inlet section 113 to also provide an insertion aid to the user. The inlet collar 130 is configured for gripping by the user during use. As such, an outer surface of the inlet collar 130 may include gripping features 131, such as protrusions, depressions, ribs, or troughs, for example, to enable grasping of the inlet collar 130 during use. In some embodiments, the inlet collar 130 includes a seal 132 closing off the distal end of the inlet collar 130 in a pre-use packaging state as further described below. In some embodiments, the seal 132 may include a film 133 sealably coupled with the inlet collar 130, and in some embodiments, the film 133 may include a pull ring 134 to facilitate removal of the film 133 from the inlet collar 130.

The catheter tube 110 also includes an outlet hub (hub) 150. The hub 150 includes gripping features 151 disposed on an outside surface of the hub 150 to enable grasping of the hub 150 during use. The 150 defines a hub chamber 155. The hub chamber 155 is in fluid communication with the catheter lumen 115, i.e., the hub chamber 155 defines the catheter lumen 115 at the proximal end 111. The hub 150 includes an outlet seal 152 at the proximal end of the hub 150.

The catheter 100 further includes a sheath 120 extending along the catheter tube 110 from the inlet collar 130 to the hub 150. The sheath 120 defines a sheath lumen 125 and the catheter tube 110 is disposed within the sheath lumen 125. The sheath lumen 125 is in fluid communication with the hub chamber 155, i.e., the sheath lumen 125 extends into the hub chamber 155. The sheath 120 (or more specifically a distal end of the sheath 120) is sealably attached to the inlet collar 130 and the sheath 120 is also sealably attached to the hub 150.

The sheath lumen 125, the catheter lumen 115 and the hub chamber 155 combine to form a closed volume and a lubricant 108 is sealably contained within the closed volume in the packaging state. The lubricant 108 is configured to enable sliding displacement of the inlet section 113 along the urethra during use.

FIG. 1B illustrates the catheter 100 in an unpackaged state, i.e., a state where the catheter 100 is removed from the outer package 104, the catheter tube 110 is uncoiled, and the inlet seal 132 and the outlet seal 152 are breached. The catheter 100 is oriented so that the lubricant 108 may flow out of the catheter lumen 115 and/or the sheath lumen 125 via the outlet hub 150.

FIGS. 1C-1E illustrate various detailed views of the outlet hub 150. FIG. 1C illustrates the hub 150 with the outlet seal 152 in the closed state. The hub 150 includes an engagement member in the form of a cap 160 rotatably coupled with a hub body 153 via a hinge 154 (e.g., a living hinge). The cap 160 is rotated into a closed position to define the outlet seal 152 in the closed state.

FIG. 1D illustrates the hub 150 with the outlet seal 152 in a breached state. As shown, the cap 160 is rotated away from hub body 153 toward an open position of the cap 160 to define an open state of the proximal end 111 of the catheter tube 110, i.e., an open end of the catheter tube lumen 115 via an open proximal end the hub chamber 155. As the sheath lumen 125 is in fluid communication with the hub chamber 155, a proximal end of the sheath lumen 125 is also open via the open proximal end the hub chamber 155. The cap 160 includes a tab (or flange) 163 protruding away from a circumference edge of the cap 160, where the tab 163 is configured to facilitate the application of an opening force by a user.

In some embodiments, the hinge 154 may bias the cap 160 toward the open position. More specifically, the hinge 154 may be configured to apply a rotating force to the cap 160 to (i) rotate the cap 160 toward the open position after the user applies the opening force to the tab 163 to rotate the cap 160 away from the closed position, and/or (ii) maintain the cap 160 in the open position. The cap 160 is generally positioned away from urine exiting the catheter tube 110 during use, when the cap 160 is in the open position.

FIG. 1E is a detailed illustration of a proximal portion of the hub 150. The cap 160 is disposed in the closed position. A portion of the hub body 153 is cut away showing a hub wall 156 of the hub body 153 in cross section. The cap 160 includes a cylindrical wall 161 coupled with an end wall 162. In the illustrated embodiment, the end wall 162 and at least a portion of the cylindrical wall 161 are disposed within the hub chamber 155 when the cap 160 is disposed in the closed position. The end wall 162 may include an end wall surface 162A that is flat or convex (i.e., not concave) to inhibit (i) a collection of lubricant 108 across the end wall 162 while draining the lubricant 108 from the catheter 100 and/or (ii) a collection of urine draining the urine from the patient during use. In other embodiments, the cap 160 may extend over the proximal end of the hub body 153 so that the cylindrical wall 161 and end wall 162 are disposed external the hub chamber 155.

The outlet seal 152 is defined by engagement of corresponding sealing members of the hub body 153 and the cap 160. The cylindrical wall 161 includes an annular rib 152A extending radially outward from the cylindrical wall 161. The hub body 153 includes an annular trough 152B extending radially outward from an inside surface of the hub wall 156. The annular rib 152A and annular trough 152B define the corresponding sealing members. The annular rib 152A and the annular trough 152B are configured to define the outlet seal 152 when the cap 160 is disposed in the closed position. In the illustrated embodiments, the annular rib 152A and annular trough 152B may also define a detent to secure the cap 160 in the closed position in the absence of the opening force applied to the cap 160. As may be appreciate by one of ordinary skill, the hub 150 may include corresponding sealing members other than the annular rib 152A and annular trough 152B. In other words, the hub 150 may include any configuration of corresponding sealing members suitable for defining the outlet seal 152.

In use, the user may remove the film 133 from the inlet collar 130 to breach the inlet seal 132. Removing the film 133 may include pulling on the pull ring 134. In use, the user may breach the outlet seal 152 by rotating the cap 160 away from the closed position. In further use, the user may re-establish the outlet seal 152 by rotating the cap 160 into the closed position.

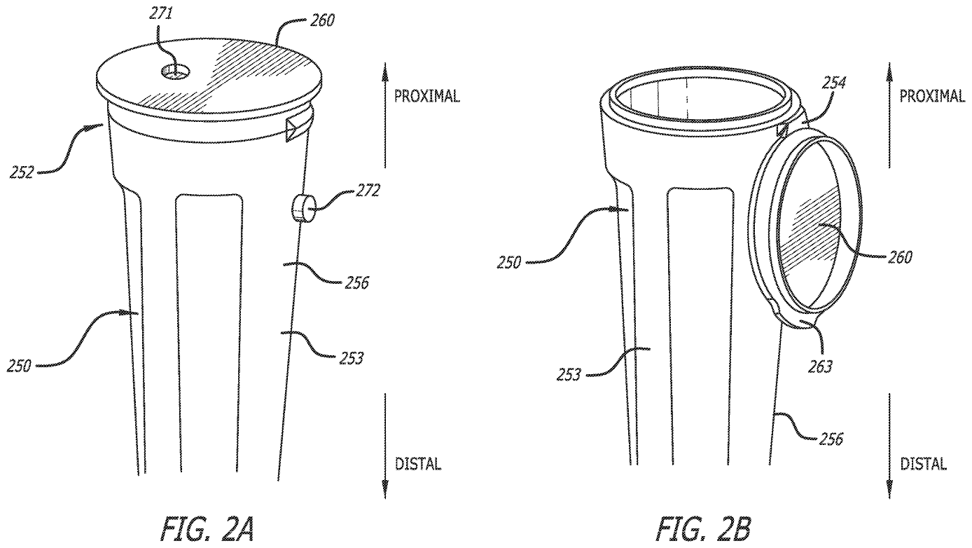

FIGS. 2A-2B illustrate another embodiment of an outlet hub (hub) 250 that may be employed with the catheter 100. The hub 250 can, in certain respects, resemble components of the hub 150 described in connection with FIGS. 1A-1E. It will be appreciated that all the illustrated embodiments may have analogous features. Accordingly, like features are designated with like reference numerals, with the leading digits incremented to “2.” For instance, the hub chamber is designated as “155” in FIGS. 1A-1E, and a hub chamber is designated as “255” in FIGS. 2A-2B. Relevant disclosure set forth above regarding similarly identified features thus may not be repeated hereafter. Moreover, specific features of the hub 150 and related components shown in FIGS. 1A-1E may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the hub 250. Any suitable combination of the features, and variations of the same, described with respect to the hub 150 and components illustrated in FIGS. 1A-1E can be employed with the hub 250 and components of FIG. 2A-2B, and vice versa. This pattern of disclosure applies equally to further embodiments depicted in subsequent figures and described hereafter.

FIG. 2A illustrates a proximal portion of the hub 250 with the outlet seal 252 in the closed state, and FIG. 2B illustrates the proximal portion of the hub 250 with the outlet seal 252 in the breached state. With reference to FIGS. 2A-2B, the hub 250 includes a cap 260 rotatably coupled with a hub body 253 via a hinge 254 (e.g., a living hinge). The cap 260 is rotated into a closed position to define the closed state of the outlet seal 252 in FIG. 2A, and the cap 260 is rotated into an open position to define the open state of the outlet seal 252 in FIG. 2B.

The hub 250 includes a cap retention mechanism to retain the cap 260 in the open position (see FIG. 2B). The hub body 253 includes a protruding member 272 disposed on a side of the hub body 253. The cap 260 includes a receiving member 271 on a top side of the cap 260. The receiving member 271 includes a depression configured to receive the protruding member 272. The receiving member 271 is positioned on the cap 160 and the protruding member 272 positioned on a side of the hub body 253 so that the receiving member 271 is positioned coincident with the protruding member 272 when the cap 260 is rotated to the open position. In other words, the receiving member 271 and the protruding member 272 are positioned so that, when the user rotates the cap 260 to the open position, the protruding member 272 is disposed within the receiving member 271.

The receiving member 271 and the protruding member 272 may be configured to define a detent configured to retain the cap 260 in the open position in the absence of a separating force applied to the cap 260. In some embodiments, the protruding member 272 may snap into the receiving member 271 when the cap 260 is rotated to the open position. The receiving member 271 and the protruding member 272 may be configured to facilitate separation of the receiving member 271 from the protruding member 272 (e.g., via the application of a separating force) to allow the user to rotate the cap 260 away from the open position toward the closed position. As may be appreciate by one of ordinary skill, other configurations of the receiving member 271 and the protruding member 272 may be consistent with retaining the cap 260 in the open position. For example, the cap 260 may include the protruding member 272 and the hub body 253 may include the receiving member 271.

In use, the user may breach the outlet seal 252 by rotating the cap 260 away from the closed position. The user may also rotate the cap 260 to a fully open position and secure the cap 260 to the wall 256 of the hub body 253 by causing the receiving member 271 to engage the protruding member 272. In further use, the user may re-establish the outlet seal 252 by rotating the cap 260 into the closed position.

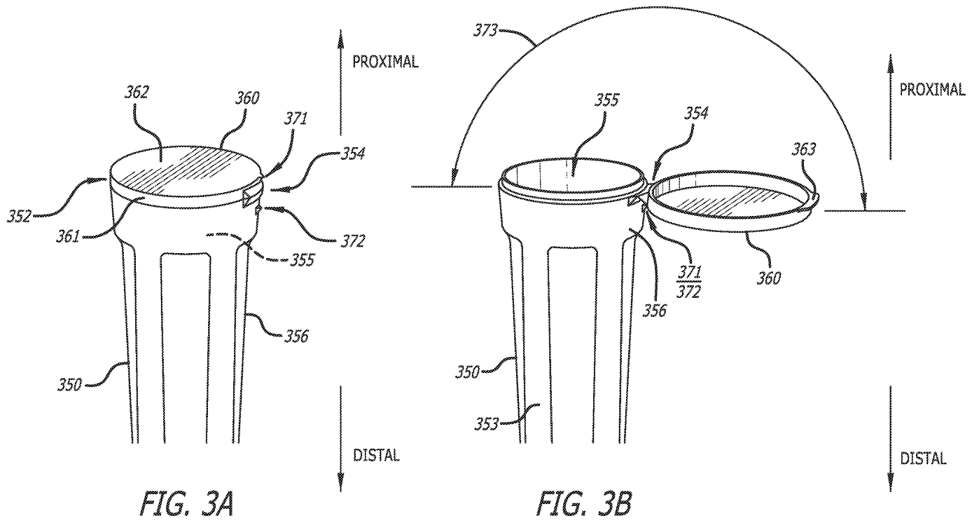

FIGS. 3A-3B illustrate another embodiment of an outlet hub (hub) 350 that may be employed with the catheter 100. FIG. 3A illustrates a proximal portion of the hub 350 with the outlet seal 352 in the closed state, and FIG. 3B illustrates the proximal portion of the hub 350 with the outlet seal 352 in the breached/open state. With reference to FIGS. 3A-3B, the hub 350 includes a cap 360 rotatably coupled with a hub body 353 via a hinge 354 (e.g., a living hinge). The cap 360 is rotated into a closed position to define the closed state of the outlet seal 352 in FIG. 3A, and the cap 360 is rotated into an open position in FIG. 3B, where the cap 360 is rotated an angle 373 away from the closed position. In some embodiments, the angle 373 may be greater than about 80 degrees, greater than about 90 degrees, greater than about 170 degrees, or about 180 degrees.

The hub 350 includes a cap retention mechanism to retain the cap 360 in the open position. The hub body 353 includes a protruding member 372 disposed on a side of the hub body 353. The cap 360 includes a protrusion 371 extending away from circumferential edge of the cap 360 at a location adjacent the hinge 354. The hub body 353 also includes a corresponding protrusion 372 extending away from the wall 356 of the hub body 353 at a location adjacent the hinge 354. The protrusion 371 and the corresponding protrusion 372 are configured to slidingly interfere with each other when the cap 360 is rotated between the closed position and the open position by the user. The protrusion 371 and the corresponding protrusion 372 are also configured so that the interference retains the cap 360 in the open position unless the user rotates the cap 360 toward the closed position. Said another way, the protrusion 371 and the corresponding protrusion 372 define a detent to maintain the cap 360 in the open position unless the user rotates the cap 360 toward the closed position.

In the illustrated embodiment, the cap 360 may extend over the proximal end of the hub body 353 so that the cylindrical wall 361 and end wall 362 are disposed external the hub chamber 355. In other embodiments, the cap 360 may be configured so that a portion of the cap 360 is disposed within the hub chamber 355 when the cap 360 is disposed in the closed position. More specifically, the cap 360 may be configured so that the end wall 362 and at least a portion of the cylindrical wall 361 are disposed within the hub chamber 355 when the cap 360 is disposed in the closed position.

In use, the user may breach the outlet seal 352 by rotating the cap 360 away from the closed position. The user may also rotate the cap 360 to the open position so that the cap 360 is retained in the open position via a cap retention mechanism. In further use, the user may re-establish the outlet seal 352 by rotating the cap 360 into the closed position.

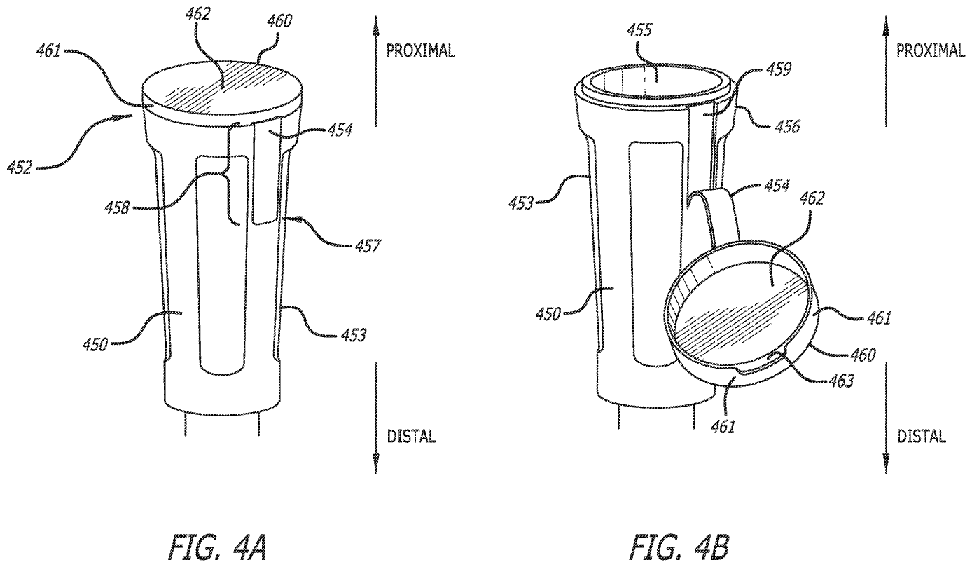

FIGS. 4A-4B illustrate another embodiment of an outlet hub (hub) 450 that may be employed with the catheter 100. FIG. 4A illustrates a proximal portion of the hub 450 with the outlet seal 452 in the closed state, and FIG. 4B illustrates the proximal portion of the hub 450 with the outlet seal 452 in the breached/open state. With reference to FIGS. 4A-4B, the hub 450 includes a cap 460 attached to a hub body 453 via a tether 454. The cap 460 is disposed in a closed position to define the closed state of the outlet seal 452 in FIG. 4A, and the cap 460 is disposed in an open position in FIG. 4B to define the open state of the outlet seal 452.

The tether 454 is attached to the cap 460 at a circumferential edge of the cap 460. The tether 454 is attached to the hub body 453 at an attachment point 457. The attachment point 457 is spaced distally away from a proximal end of the hub body 453 to define a length of 458 of the tether 458. In some embodiments, the wall 456 of the hub body 453 may include a trough 459, where the trough 459 is configured to receive the tether 454 therein when the cap 460 is disposed in the closed position.

In the illustrated embodiment, the cap 460 may extend over the proximal end of the hub body 453 so that the cylindrical wall 461 and end wall 462 are disposed external the hub chamber 455. In other embodiments, the cap 460 may be configured so that a portion of the cap 460 is disposed within the hub chamber 455 when the cap 460 is disposed in the closed position. More specifically, the cap 460 may be configured so that the end wall 462 and at least a portion of the cylindrical wall 461 are disposed within the hub chamber 455 when the cap 460 is disposed in the closed position.

In some embodiments, the tether 454 may be configured to bias the cap 460 toward the open position. More specifically, the tether 454 may be configured to maintain the cap 460 at a position away from the open proximal end of the hub body 453 when the cap 460 is not disposed in the closed position. Biasing the cap 460 at a position away from the open proximal end of the hub body 453 when the cap 460 is not disposed in the closed position may inhibit contact of the cap 460 with urine draining from the outlet hub 450 during use.

In use, the user may breach the outlet seal 452 by displacing the cap 460 away from the closed position. In further use, the user may re-establish the outlet seal 452 by replacing the cap 460 in the closed position.

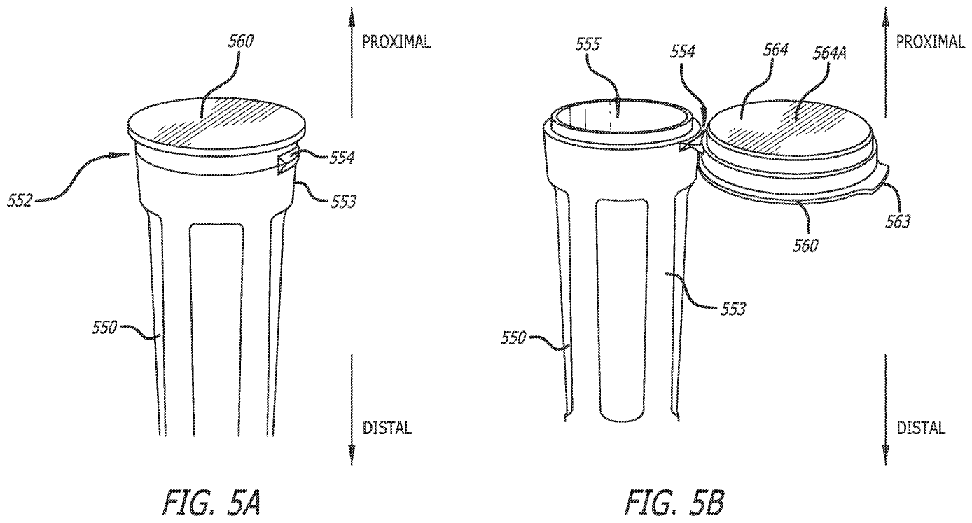

FIGS. 5A-5B illustrate another embodiment of an outlet hub (hub) 550 that may be employed with the catheter 100. FIG. 5A illustrates a proximal portion of the hub 550 with the outlet seal 552 in the closed state, and FIG. 5B illustrates the proximal portion of the hub 550 with the outlet seal 552 in the breached/open state. With reference to FIGS. 5A-5B, the hub 550 includes a cap 560 rotatably coupled with a hub body 553 via a hinge 554 (e.g., a living hinge). The cap 560 is disposed in a closed position to define the closed state of the outlet seal 552 in FIG. 5A, and the cap 560 is disposed in an open position in FIG. 5B to define the open state of the outlet seal 552.

In some embodiments, the hinge 554 may be configured to bias the cap 560 toward the open position. More specifically, the hinge 554 may be configured to apply a rotating force to the cap 560 to (i) rotate the cap 560 toward the open position after the user applies an opening force to the tab 563 to rotate the cap 560 away from the closed position, and/or (ii) maintain the cap 560 in the open position.

The hub 550 includes a plug member 564 attached to an underside of the cap 560 such that the plug member 564 is disposed within the chamber 555 when the cap 560 is disposed in the closed position. The plug member 564 may be formed of a soft material, such as an elastomeric material, for example. The plug member 564 may cover portions of the cap 560 (e.g., pockets, depressions, inside corners, and the like) that may, in the absence of the plug member 564, collect lubricant and/or urine. In some embodiments, the plug member 564 may engage the hub body 553 (or more specifically an inside surface of the hub body 553) to define (or partially define) the outlet seal 552. An underside 564A of the plug member 564 may be flat or convex (i.e., not concave).

In use, the user may breach the outlet seal 552 by displacing the cap 560 away from the closed position. In further use, the user may re-establish the outlet seal 552 by replacing the cap 560 in the closed position.

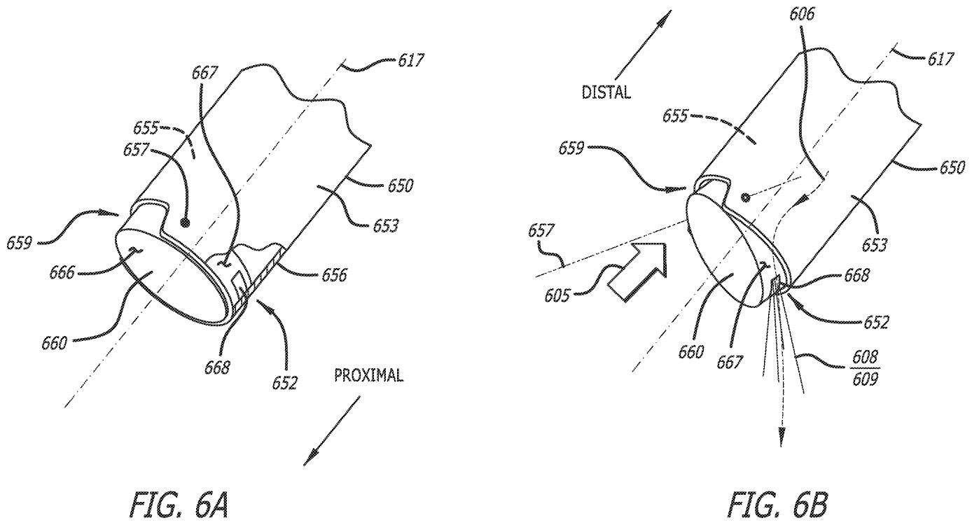

FIGS. 6A-6B illustrate another embodiment of an outlet hub (hub) 650 that may be employed with the catheter 100. FIG. 6A illustrates a proximal portion of the hub 650 with the outlet seal 652 in the closed state, and FIG. 6B illustrates the proximal portion of the hub 650 with the outlet seal 652 in the breached/open state. With reference to FIGS. 6A-6B, the hub 650 includes a cap 660 pivotably coupled with a hub body 653 so as to pivot about an axis of rotation 657, where the axis of rotation 657 passes through the hub body 653 and the cap 660. The cap 660 is pivoted to a closed position to define the closed state of the outlet seal 652 in FIG. 6A, and the cap 660 is pivoted to an open position in FIG. 6B to define the open state of the outlet seal 652.

The cap 660 is generally takes the form of a plug disposed within the hub chamber 655. The cap 660 includes a circumferential wall 667 coupled with an end wall 666. In some embodiments, the end wall 666 is oriented perpendicular to a longitudinal axis 617 of the outlet hub 650. In the closed state, the circumferential wall 667 sealably engages the wall 656 of the hub body 653 to define the outlet seal 652. The cap 660 defines a fluid flow path 606 extending through the cap 660 from bottom side of the cap to a port 668 extending through the circumferential wall 667. The port 668 is positioned so that a hub wall 656 extends across (i.e., covers) the port 668 when the cap 660 is pivoted to the closed position.

The cap 660 is configured to pivot toward the open position (FIG. 6B) in response to a force 605 applied to the cap 660 by the user. When the cap 660 is pivoted to the open position, the port 668 is disposed proximal the proximal end of the hub body 653 to define the fluid flow path out 606 of the hub chamber 655. As such, when the cap 660 is pivoted to the open position, lubricant 608 and/or urine 609 may exit the chamber 655.

In some embodiments, the hub body 653 may include a finger relief 659. The finger relief may enable the user to apply the force 605 to pivot the cap 660 from the closed position to the open position.

In use, the user may breach the outlet seal 652 by pivoting the cap 660 away from the closed position to uncover the port 668. In further use, the user may re-establish the outlet seal 652 by pivoting the cap 660 back to the closed position to recover the port 668.

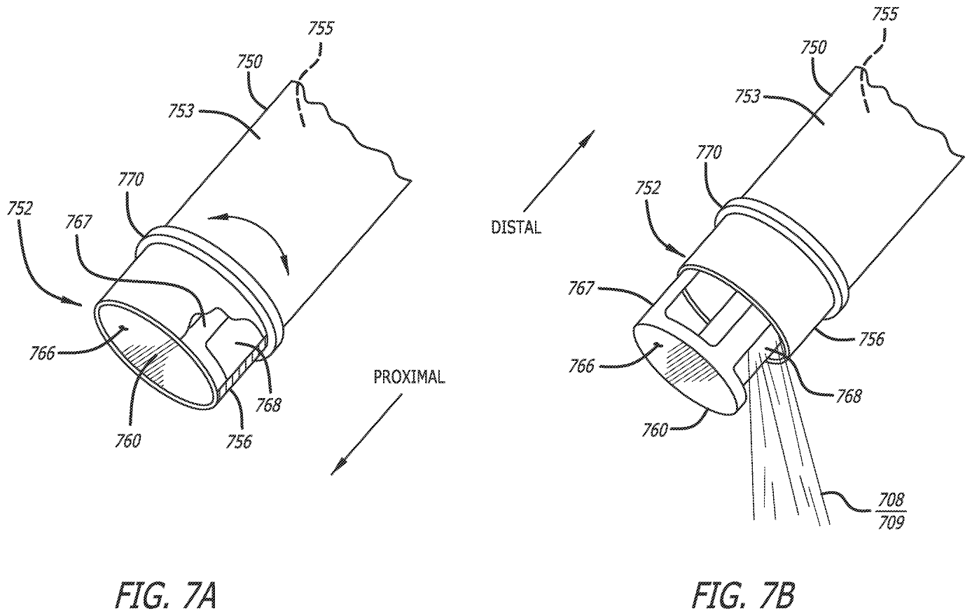

FIGS. 7A-7B illustrate another embodiment of an outlet hub (hub) 750 that may be employed with the catheter 100. FIG. 7A illustrates a proximal portion of the hub 750 with the outlet seal 752 in the closed state, and FIG. 7B illustrates the proximal portion of the hub 750 with the outlet seal 752 in the breached/open state. With reference to FIGS. 7A-7B, the hub 750 includes a cap 760 coupled with a hub body 753 such that the cap 760 is longitudinally positionable with respect to the hub body 753. The cap 760 is disposed in a closed position to define the closed state of the outlet seal 752 in FIG. 7A, and the cap 760 is disposed in an open position in FIG. 7B to define the open state of the outlet seal 752, where the open position is proximal the closed position.

The cap 760 is generally disposed within the hub chamber 755. The cap 760 includes a cylindrical wall 767 coupled with an end wall 766. In the closed state, the cylindrical wall 767 sealably engages the wall 756 of the hub body 753 to define the outlet seal 752. The cap 760 includes one or more ports 768 extending through the cylindrical wall 767. The ports 768 are positioned so that the hub wall 756 extends across (i.e., covers) the ports 768 when the cap 760 is disposed in the closed position. In the open position, the cap 760 extends proximally beyond a proximal end of the hub body 753 to uncover the ports 768. As such, when the cap 760 is longitudinally displaced to the open position, lubricant 708 and/or urine 709 may exit the chamber 755 through the ports 768.

The hub 750 includes a ring 770 surrounding the hub body 753. The ring 770 is rotationally coupled with the hub body 753. The ring 770 is also operationally coupled with the cap 760 such that rotation of the ring 770 in one direction causes the cap 760 to proximally displace away from the closed position to the open position. Similarly, rotation of the ring 770 in the opposite direction causes the cap 760 to distally displace away from the open position to the closed position.

In use, the user may breach the outlet seal 752 by rotating the ring 770 in one direction to proximally extend the cap 760 to uncover the ports 768. In further use, the user may re-establish the outlet seal 752 by rotating the ring 770 in the opposite direction to distally displace the cap 760 into the closed position to recover the ports 768.

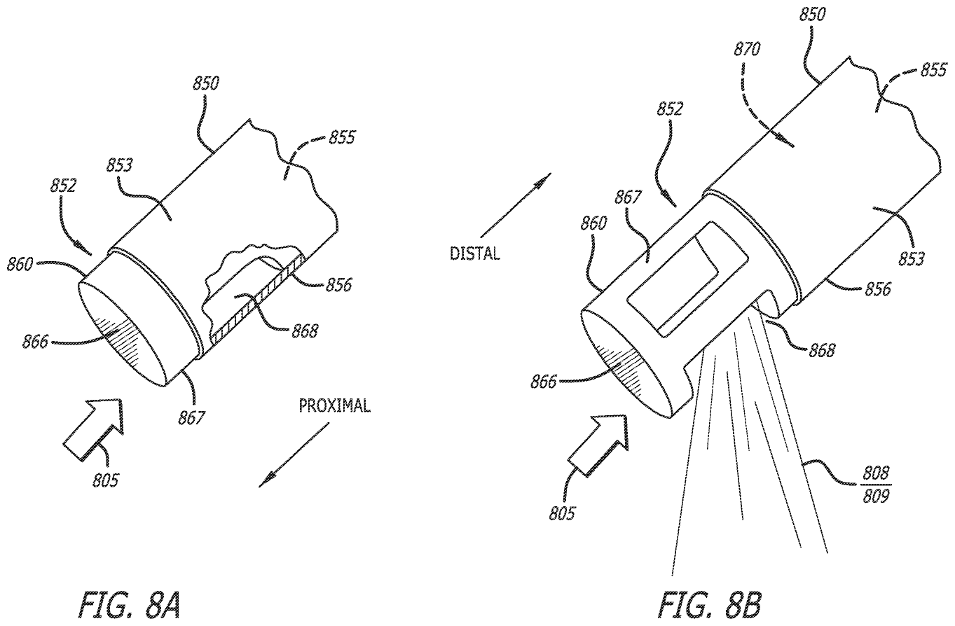

FIGS. 8A-8B illustrate another embodiment of an outlet hub (hub) 850 that may be employed with the catheter 100. FIG. 8A illustrates a proximal portion of the hub 850 with the outlet seal 852 in the closed state, and FIG. 8B illustrates the proximal portion of the hub 850 with the outlet seal 852 in the breached state. With reference to FIGS. 8A-8B, the hub 850 includes a cap 860 coupled with a hub body 853 such that the cap 860 is longitudinally positionable with respect to the hub body 853. The cap 860 is disposed in a closed position to define the closed state of the outlet seal 852 in FIG. 8A, and the cap 860 is disposed in an open position in FIG. 8B to define the open state of the outlet seal 852, where the open position is proximal the closed position.

The cap 860 is generally disposed within the hub chamber 855 such that a proximal end portion of the cap 860 extends beyond a proximal end of the hub body 853. The cap 860 includes a cylindrical wall 867 coupled with an end wall 866. In the closed state, the cylindrical wall 867 sealably engages the hub wall 856 of the hub body 853 to define the outlet seal 852. The cap 860 includes one or more ports 868 extending through the cylindrical wall 867. The ports 868 are positioned so that the hub wall 856 extends across (i.e., covers) the ports 868 when the cap 860 is disposed in the closed position. In the open position, the cap 860 extends further beyond a proximal end of the hub body 853 to uncover the ports 868. As such, when the cap 860 is longitudinally displaced to the open position, lubricant 808 and/or urine 809 may exit the chamber 855 through the ports 868.

The hub 850 includes a latching mechanism 870 operatively coupled between the cap 860 and the hub body 853. The latching mechanism 870 facilitates selective positioning of the cap 860 between the closed position and the open position. The latching mechanism 870 biases the cap 860 in the proximal direction, and the latching mechanism 870 selectively limits the position of the cap 860 (i.e., prevents proximal displacement of the cap 860) at the closed position and the open position. The latching mechanism 870 is configured to transition the cap 860 from the closed position to the open position in response to a force 805 applied to the cap 860 by the user. Similarly, the latching mechanism 870 is configured to transition the cap 860 from the open position to the closed position in response to the force 805 applied to the cap 860 by the user. Said another way, the user may apply the force 805 to the cap 860 when the cap 860 is in the closed position. In response, the cap 860 self-displaces from the closed position to the open position upon removal of the force 805. Similarly, the user may apply the force 805 to the cap 860 when the cap 860 is in the open position to displace the cap 860 to the closed position. In response, the latching mechanism 870 limits proximal displacement of the cap 860 in the closed position upon removal of the force 805.

In use, the user may breach the outlet seal 852 by distally pressing the cap 860 and the allowing the cap 860 to self-displace to the open position to uncover the ports 868. In further use, the user may re-establish the outlet seal 852 by distally pressing the cap 860 to the closed position to recover the ports 868.

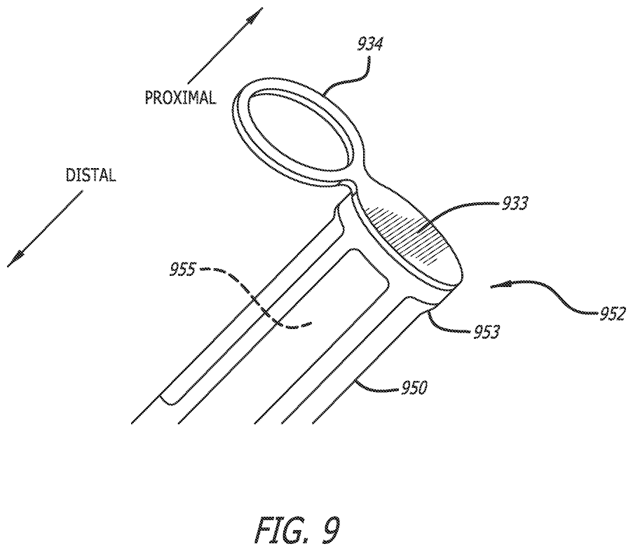

FIG. 9 illustrates another embodiment of an outlet hub (hub) 950 that may be employed with the catheter 100. FIG. 9 illustrates a proximal portion of the hub 950 with the outlet seal 952 in the closed state. The outlet seal 952 includes a film 933 sealably coupled with the hub body 953 to close off the proximal end of the hub chamber 955. The film 933 may be attached to the hub body 953 via a heat sealing process. The film 933 may include a pull ring 934 to enable the user to remove the film 933 from the hub body 953.

In use, the user may breach the outlet seal 952 by removing the film 933. In some embodiments, removing the film may include pulling on the pull ring 934.

Methods of using the catheter to drain urine from the bladder may include all or any subset of the following steps or process as self-performed by the user. A step may include the user removing the catheter from the outer package.

Another step may include the user breaching the inlet seal and/or the outlet seal.

Another step may include draining the lubricant from the lumen of the catheter tube. Draining the lubricant may include transitioning the catheter tube from a looped shape toward an extended shape. Draining the lubricant may also include orienting the catheter tube toward a vertical attitude so that gravity may induce the lubricant to drain from lumen toward the proximal end or the distal end.

Another step may include orienting and/or positioning the distal end of the catheter tube in relation the urethra of the user. Another step may include inserting the distal tip of the inlet section into the urethra. Another step may include advancing the inlet section along the urethra to the bladder. Another step may include draining the urine from the bladder. In some embodiments, draining the urine from the bladder may include placing the proximal end of the tube at a lower vertical position than the distal end to cause the urine to flow: (i) into the openings, (ii) proximally along the lumen, and (iii) out of the proximal end. In some embodiments, may draining the urine from the bladder include positioning the proximal end of the tube over a urine receptacle, such as a toilet, for example to cause the urine to enter the receptacle. Another step may include withdrawing the inlet section from the urethra.

Another step may include re-establishing a seal at the proximal end of the catheter. Another step may include replacing the catheter into the outer package. Another step may include disposing the catheter in a trash receptacle.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary catheter comprising:
a catheter tube defining a catheter lumen extending between distal end and a proximal end, the catheter tube configured for insertion into a patient bladder;
a sheath defining a sheath lumen extending along the catheter tube, the catheter tube disposed within the sheath lumen;
an outlet hub coupled with the catheter tube at the proximal end, the outlet hub including a hub body defining a hub chamber, the hub chamber defining the catheter lumen extending through the outlet hub; and
an engagement member coupled with the outlet hub, the engagement member selectively transitionable between:
a closed state wherein a fluid is prevented from exiting the catheter lumen at the proximal end, and
an open state wherein the fluid is allowed to exit the catheter lumen at the proximal end.

2. The catheter of claim 1, further comprising a lubricant disposed within the catheter lumen.

3. The catheter of claim 2, wherein the lubricant is further disposed within the sheath lumen in contact with an outer surface of the catheter tube.

4. The catheter of claim 1, wherein the fluid includes the lubricant.

5. The catheter of claim 1, wherein:
the engagement member includes a lid coupled with the outlet hub via a hinge,
the lid is rotatable between a closed position defining the closed state and an open position defining the open state,
the lid extends across a proximal end of the outlet hub in the closed position, and
the lid is rotated away from the proximal end of the outlet hub in the open position.

6. The catheter of claim 5, wherein, in the open position, the lid is rotated at least 90 degrees away from the closed position.

7. The catheter of claim 5, wherein, in the open position, the lid is rotated at least 180 degrees away from the closed position.

8. The catheter of claim 5, wherein the lid is rotatably biased toward the open position via the hinge.

9. The catheter of claim 5, wherein:
the lid includes a first protrusion extending away from a circumferential edge of the lid;
the outlet hub includes a corresponding second protrusion extending away from a side wall of the outlet hub, the corresponding second protrusion positioned adjacent the hinge; and
an interference between the corresponding second protrusion and the first protrusion defines a detent to maintain the lid in the open position.

10. The catheter of claim 5, wherein:
the outlet hub includes a protrusion extending away from a side wall of the outlet hub,
the lid includes a protrusion receiving member disposed on a top side of the lid, the receiving member configured to inhibit separation of the protrusion from the protrusion receiving member in the absence of a separating force, and
the protrusion is inserted into the protrusion receiving member to maintain the lid in the open position.

11. The catheter of claim 5 wherein:
the lid includes a plug disposed on an underside of the lid, and
at least a portion of the plug is disposed within the hub chamber when the lid is in the closed position.

12. The catheter of claim 11, wherein the plug is formed of an elastomeric material.

13. The catheter of claim 1, wherein:
the engagement member includes a lid coupled with the outlet hub via a tether,
the lid is positionable between a closed position defining the closed state and an open position defining the open state,
the lid extends across a proximal end of the outlet hub in the closed position,
the lid is positioned away from the proximal end of the outlet hub in the open position, and
the tether biases the lid away from the closed position toward the open position.

14. The catheter of claim 1, wherein:
the engagement member includes a plug cap disposed within the hub chamber,
the plug cap includes a fluid flow path extending through the plug cap between an underside of the plug cap and one or more side ports located along a cylindrical side wall of the plug cap,
the plug cap is positionable between a closed position defining the closed state and an open position defining the open state,
the one or more side ports are disposed within the hub chamber when plug cap is in the closed position such that a side wall of the outlet hub sealably covers the one or more side ports, and
the one or more side ports are disposed proximally beyond the side wall of the outlet hub when the plug cap is in the open position.

15. The catheter of claim 14, wherein:
the plug cap is coupled with the outlet hub so as to be tiltable with respect to the outlet hub about an axis of rotation extending laterally through the plug cap and the outlet hub.
the plug cap is oriented such that a proximal end face of the plug cap is perpendicular to a longitudinal axis of the outlet hub when the plug cap is in the closed position, and
the plug cap is tilted such that the proximal end face is rotated away from the perpendicular orientation when the plug cap is in the open position

16. The catheter of claim 14, wherein the plug cap is longitudinally positionable between the closed position and the open position.

17. The catheter of claim 15, wherein:
the outlet hub includes a ring extending circumferentially around an exterior of the hub body, and
the ring is operatively coupled between the plug cap and the hub body such that:
rotation of the ring in one direction with respect to the hub body causes the plug cap to proximally displace from the closed position to the open position, and
rotation of the ring in the opposite direction with respect to the hub body causes the plug cap to distally displace from the open position to the closed position.

18. The catheter of claim 14, wherein the outlet hub includes a latching mechanism operatively coupled between the plug cap and the hub body, such that:
the latching mechanism positions the plug cap at the open position when a distally oriented force is (i) manually applied to the plug cap and (ii) released from the plug cap when the plug cap is in the closed position, and
the latching mechanism positions the plug cap at the closed position when the distally oriented force is (i) manually applied to the plug cap and (ii) released from the plug cap when the plug cap is in the open position.

19-29. (canceled)
